(19) United States
(12) Patent Application Publication
(10) Pub. No.: US 2013/0030497 A1
(43) Pub. Date: Jan. 31, 2013

(54) NERVE STIMULATOR FOR REDUCED MUSCLE FATIGUE

(71) Applicants: Mustafa Karamanoglu, Fridley, MN (US); Vincent E. Splett, Apple Valley, MN (US)

(72) Inventors: Mustafa Karamanoglu, Fridley, MN (US); Vincent E. Splett, Apple Valley, MN (US)

(73) Assignee: Medtronic, Inc., Minneapolis, MN (US)

(21) Appl. No.: 13/192,195

(22) Filed: Jul. 27, 2011

(51) Int. Cl. A61N 1/36; A61B 5/08

(52) U.S. Cl. 607/42

(57) ABSTRACT

A system and associated method deliver a respiration therapy. Stimulation pulse trains are delivered to activate a first portion of a patient's diaphragm at an established intended induced respiration rate during a time interval. Stimulation pulse trains are delivered to activate a second portion of a patient's diaphragm at a second rate during the time interval, the second rate being different than the first rate.

### TECHNICAL FIELD

The disclosure relates generally to implantable medical devices and, more particularly, to nerve stimulation with reduced muscle fatigue.

### BACKGROUND

Activation of the diaphragm through direct stimulation of the diaphragm or stimulation of the right and/or left phrenic nerves to cause contraction of the diaphragm has been proposed for treating respiratory insufficiency, e.g. in paralysis, apnea, or other respiratory conditions. Phrenic nerve stimulation may be achieved using nerve electrodes implanted in proximity to the phrenic nerve in an open surgical approach. A less invasive approach for implanting electrodes positioned in proximity to a phrenic nerve is a transvenous approach. Activation of the diaphragm to induce respiration through electrical stimulation can lead to fatigue of the diaphragm and a reduced or absent respiratory response. A need remains for an apparatus and method for effectively delivering respiration therapy.

### BRIEF DESCRIPTION OF THE DRAWINGS

FIG. 1 is a schematic view of an implantable medical device (IMD) system for delivering phrenic nerve stimulation.

FIG. 2 is a schematic view of an IMD system for delivering phrenic nerve stimulation according to an alternative embodiment.

FIG. 3 is a schematic view of an IMD system for delivering phrenic nerve stimulation according to another alternative embodiment.

FIG. 4 is a functional block diagram of an IMD system that may include any of the leads configured for the implant locations shown in FIGS. 1 through 3.

FIG. 5 is a flow chart of a method for controlling phrenic nerve stimulation for delivering a respiration therapy according to one embodiment.

FIG. 6 is a timeline showing pulse trains delivered to the left phrenic nerve and pulse trains delivered to the right phrenic nerve.

FIG. 7 is a timeline illustrating an alternative method for controlling activation of the diaphragm to reduce fatigue.

FIG. 8 is a flow chart of an alternative method for controlling bilateral activation of the diaphragm.

FIG. 9 is a flow chart of a method for establishing a target range for a respiratory status parameter and controlling alternation of bilateral and unilateral activation of the diaphragm according to one embodiment.

### DETAILED DESCRIPTION

In the following description, references are made to illustrative embodiments. It is understood that other embodiments may be utilized without departing from the scope of the disclosure.

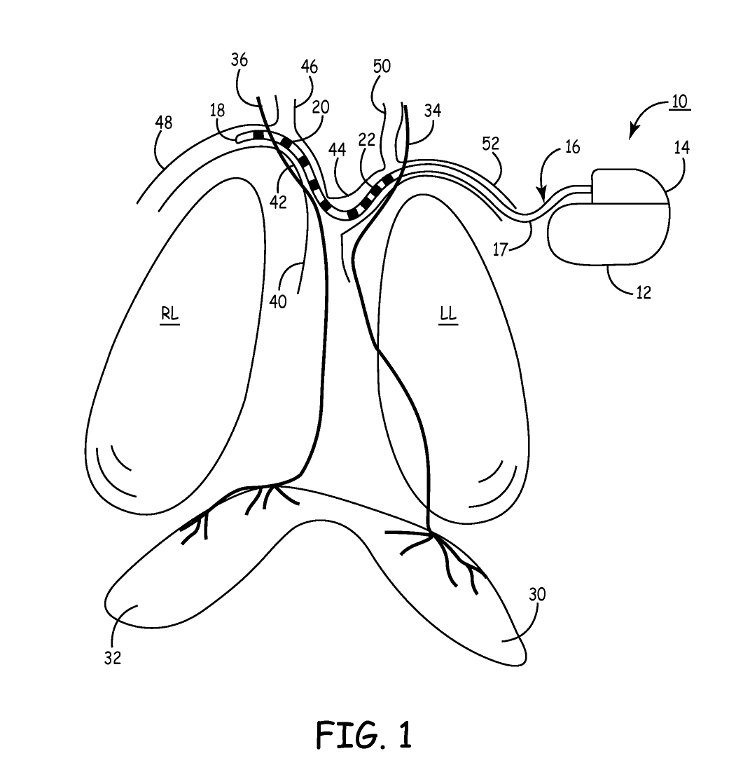

FIG. 1 is a schematic view of an implantable medical device (IMD) system for delivering phrenic nerve stimulation. IMD 10 includes a housing 12 enclosing electronic circuitry (not shown) included in IMD 10 and a connector block 14 having at least one connector bore for receiving at least one medical electrical lead 16. Connector bores included in block 14 provide electrical connection between electrodes carried by lead 16 and IMD internal electronic circuitry.

In FIG. 1, the left phrenic nerve 34 and the right phrenic nerve 36 are shown innervating the respective left diaphragm 30 and right diaphragm 32. The anatomical locations of the left phrenic nerve 34, the right phrenic nerve 36 and other anatomical structures shown schematically in the drawings presented herein are intended to be illustrative of the approximate and relative locations of such structures. These structures are not necessarily shown in exact anatomical scale or location. The left phrenic nerve 34 is shown schematically to extend in close proximity to the left internal jugular vein (LJV) 50, the left subclavian vein (LSV) 52, and the left innominate vein (LIV) 44, also referred to as the left brachiocephalic vein.

The anatomical location of the right phrenic nerve 36 is shown schematically to extend in close proximity to the right internal jugular vein (RJV) 46, the right subclavian vein (RSV) 48, the right innominate vein (RIV) 42 (also referred to as the right brachiocephalic vein), and the superior vena cava (SVC) 40.

Lead 16 is a multipolar lead carrying proximal electrodes 22 and distal electrodes 20 at or near the distal end 18 of lead 16. In one embodiment, at least one proximal bipolar pair of electrodes is provided for stimulating the left phrenic nerve 34 and at least one distal bipolar pair of electrodes is provide for stimulating the right phrenic nerve 36. In various embodiments, two or more electrodes may be positioned along spaced apart locations adjacent the distal tip of lead 16 from which at least one pair of electrodes is selected for delivering stimulation to the right phrenic nerve 36. Additionally, two or more electrodes may be positioned along spaced apart locations proximally from the distal electrodes 20 from which at least one pair of electrodes is selected for delivering stimulation to the left phrenic nerve 34.

Lead 16 includes an elongated lead body 17, which may have a diameter in the range of approximately 2 French to 8 French, and typically approximately 4 French to approximately 6 French. The lead body 17 carries the electrodes 20 and 22 which are electrically coupled to electrically insulated conductors extending from individual respective electrodes 20 and 22 to a proximal connector assembly adapted for connection to IMD connector block 14. Lead 16 may be provided with a fixation element for fixing the position of the lead once a desired implant location is identified. Exemplary leads that can be useful for the present disclosure include U.S. Pat. No. 5,922,014, U.S. Pat. No. 5,628,778, U.S. Pat. Nos. 4,497,326, 5,443,492, or U.S. Pat. No. 7,860,580 such that electrodes are added and/or spaced apart in a manner similar to that disclosed in the figures of the present application, all of which are incorporated by reference in their entirety. Additional lead and electrode configurations that may be adapted for use with the present disclosure by adjusting lead shape, length, electrode number and/or electrode to effectively provide phrenic nerve stimulation as described herein are generally disclosed in U.S. Pat. No. 7,031,777, U.S. Pat. No. 6,968,237, and US Publication No. 2009/0270729, all of which are incorporated herein by reference in their entirety.

In one embodiment, distal tip 18 of lead 16 is advanced to a location along the RIV 42 and further along the RSV 48 or the RJV 46 to position distal electrodes 20 in operative relation to right phrenic nerve 36 for delivering stimulation pulses to nerve 36 to activate the right diaphragm 32. The proximal electrodes 22 may be appropriately spaced from distal electrodes 20 such that proximal electrodes 22 are positioned along the LIV 44 and/or along the junction of the LSV 52 and LJV 50 for delivering stimulation pulses to the left phrenic nerve 34 to activate the left diaphragm 30.

In various embodiments, lead 16 may carry four or more electrodes spaced at selected distances to provide at least one pair near a distal lead tip 18 for right phrenic nerve stimulation and at least one pair more proximally for left phrenic nerve stimulation. In other embodiments, lead 16 may carry multiple electrodes spaced equally along a portion of the body of lead 16 such that any pair may be selected for right phrenic nerve stimulation and any pair may be selected for left phrenic nerve stimulation based on the relative locations of the electrodes from the nerves.

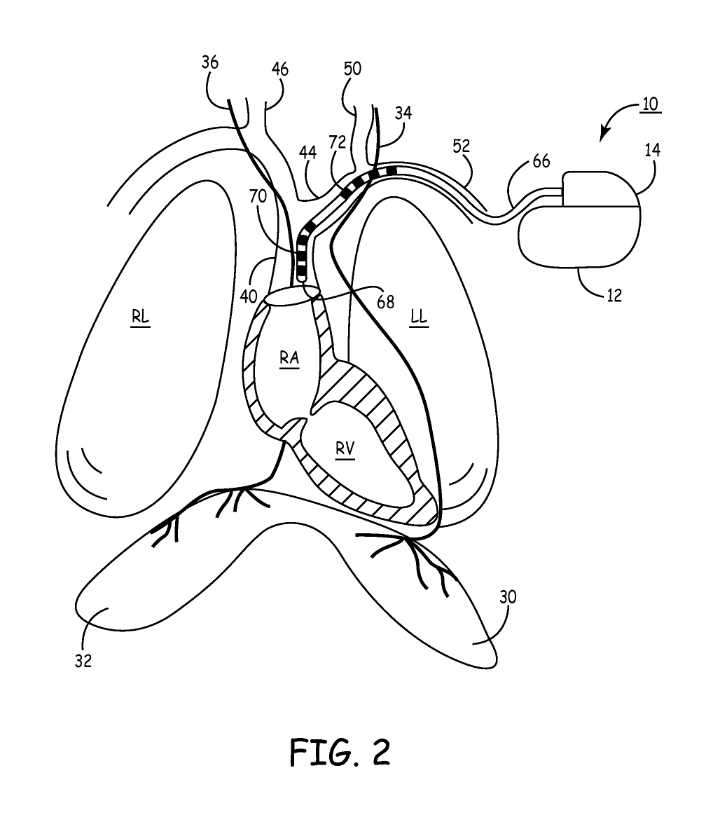

FIG. 2 is a schematic view of an IMD system for delivering phrenic nerve stimulation according to an alternative embodiment. In FIG. 2, the right atrium (RA) and the right ventricle (RV) are shown schematically in a partially cut-away view. The right phrenic nerve 36 extends posteriorly along the SVC 40, the RA and the inferior vena cava (IVC) (not shown in FIG. 2). The left phrenic nerve 34 normally extends along a left lateral wall of the left ventricle (not shown). The SVC 40 enters the RA. A lead 66 is coupled to IMD 10 via connector block 14. Lead 66 carries multiple electrodes that may be spaced apart into a plurality of distal electrodes 70 located near distal lead tip 68 and a plurality of proximal electrodes 72. The distal tip 68 of lead 66 is advanced into SVC 40 to position distal electrodes 70 in the SVC for stimulating the right phrenic nerve 36, which extends posteriorly to SVC 40 and the RA. The proximal electrodes 72 are used to stimulate the left phrenic nerve 34, e.g. along the LIV 44 or junction of the LJV 50 and LSV 52.

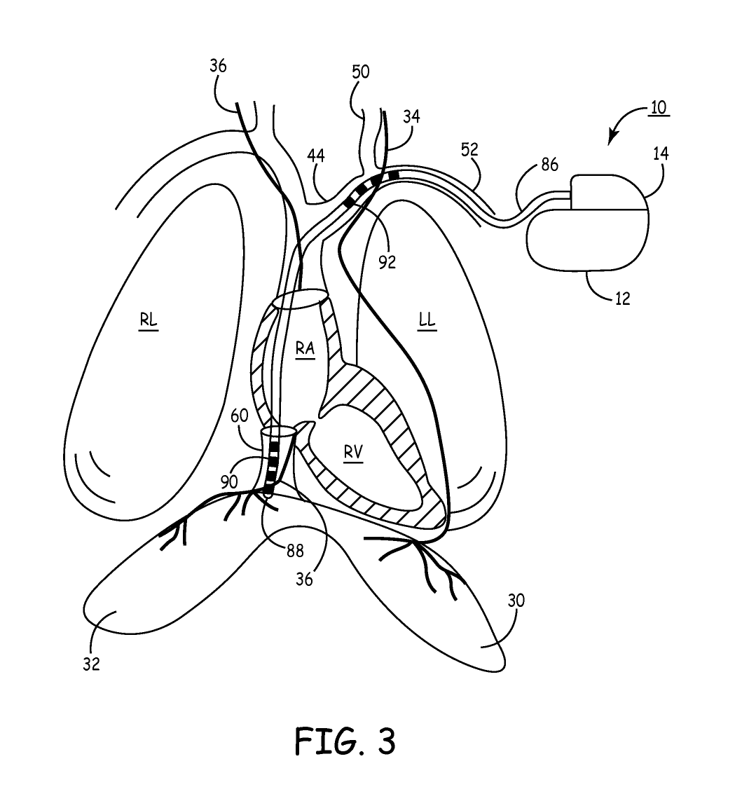

FIG. 3 is a schematic view of an IMD system for delivering phrenic nerve stimulation according to another alternative embodiment. In FIG. 3, the inferior vena cava (IVC) 60, which empties into the RA, is shown schematically. In this embodiment, lead 86 extends from IMD connector block 14 to the IVC 60 to position electrodes 90 adjacent distal lead tip 88 in the IVC 60 adjacent the right phrenic nerve 36 near the level of the diaphragm, e.g. approximately at the height of the eighth thoracic vertebra (T8) (not shown). Proximal electrodes 92 are positioned proximally along lead 86 for positioning along the LIV 44 or the junction of the LJV 50 and LSV 52 for providing stimulation to the left phrenic nerve 34. Electrodes used for stimulating the right phrenic nerve and electrodes used for stimulating the left phrenic nerve are shown configured along a common lead in FIGS. 1 through 3. In alternative embodiments it is contemplated that two leads, one for stimulating the left and one for stimulating the right phrenic nerve, may be provided separately.

The housing 12 of IMD 10 may be provided as an indifferent electrode for use in combination with any of the lead-based electrodes shown in FIGS. 1 through 3 for some monitoring purposes. As will be further described below, the electrodes included in an IMD system for delivering a respiration therapy may additionally be used for sensing impedance signals. In some embodiments, the housing 12 may provide an indifferent electrode for delivering a drive current during thoracic impedance measurements or used in a measurement pair for monitoring thoracic impedance.

It is further recognized that additional leads and electrodes may be included in an IMD system capable of delivering phrenic nerve stimulation or more generally diaphragm stimulation. For example, IMD 10 may be coupled to cardiac leads, which may be subcutaneous leads, transvenous leads positioned in or along a heart chamber, or epicardial leads. IMD 10 may incorporate sensing electrodes along housing 12. IMD 10 may be provided specifically for delivering phrenic nerve stimulation (with associated monitoring of sensed signals for controlling the phrenic nerve stimulation) or may include other therapy delivery capabilities such as cardiac pacing (e.g. for bradycardia pacing, cardiac resynchronization in therapy, or anti-tachycardia pacing) cardioversion/defibrillation shocks, drug delivery or the like. As such, the IMD system may include other leads, electrodes and/or catheters not shown in FIGS. 1-3 related to other IMD functions.

In some embodiments, electrodes used for delivering phrenic nerve stimulation could be carried by leads that additionally carry cardiac pacing, sensing and/or defibrillation electrodes. In other embodiments, sensing electrodes carried by cardiac leads may be used for sensing EGM signals to control the timing of phrenic nerve stimulation.

In FIGS. 1 through 3, IMD 10 is shown in a left pectoral position such that it is the distal electrodes, e.g. electrodes 20, 70 or 90, that are positioned in operative relation to the right phrenic nerve 36 and the proximal electrodes, e.g. electrodes 22, 72 or 92, that are positioned in operative relation to the left phrenic nerve 34. Depending on the implanted configuration, lead 16 may be positioned entering a vein from a right venous approach such that it is the distal electrodes that are positioned for left phrenic nerve stimulation and the proximal electrodes that are positioned for right phrenic nerve stimulation. For example, IMD 10 may be implanted in a pocket along a right pectoral position, along a right or left abdominal position, centrally, or other implant location. The IMD implant location may determine whether it is the proximal electrodes or the distal electrodes that are positioned for stimulating the right or the left phrenic nerves, when the electrodes are all carried by a single phrenic nerve stimulation lead.

For example, a right-sided implantation of IMD 10 could include distal electrodes positioned along the LIV 44 for left phrenic nerve stimulation and proximal electrodes positioned for right phrenic nerve stimulation along the RIV 42 or junction of the RSV 48 and RJV 46.

IMD systems and associated methods described herein relate to controlling phrenic nerve stimulation to reduce diaphragm fatigue. It is recognized that stimulation of the phrenic nerves directly using a nerve electrode, stimulation of the diaphragm directly, as well as stimulation of other nerves or muscle, could employ the stimulation methods described herein for reducing muscle fatigue.

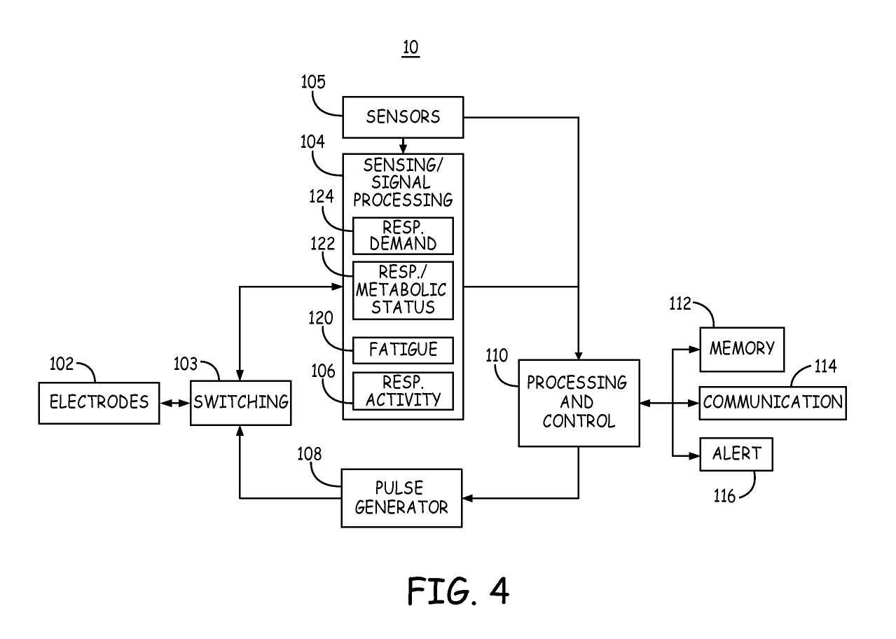

FIG. 4 is a functional block diagram of an IMD system that includes IMD 10 and may include any of the leads configured for the implant locations shown in FIGS. 1 through 3 for phrenic nerve stimulation. Electrodes 102 may include the electrodes carried by one or more transvenous phrenic nerve stimulation leads, electrodes carried by other leads such as cardiac leads, electrodes incorporated along the housing of IMD 10, or electrodes implanted subcutaneously or submuscularly such as electrodes positioned for stimulating the diaphragm directly, nerve branches innervating the diaphragm, or for sensing diaphragm EMG signals. Reference is made, for example, to U.S. Pat. No. 5,524,632 (Stein, et al.), hereby incorporated herein by reference in its entirety.

Electrodes 102 may be selectively coupled via switching circuitry 103 to sensing and signal processing circuitry 104 for monitoring a respiratory response to phrenic nerve stimulation pulses, determining a need to adjust stimulation parameters, and for detecting diaphragm fatigue. Electrodes 102 are further coupled to pulse generator 108 via switching circuitry 103. Electrodes and electrode polarity for delivering phrenic nerve stimulation pulses generated by pulse generator 108 may be selected via switching circuit 103. In alternative embodiments, phrenic nerve stimulation electrodes and sensing electrodes, which may be dedicated or shared electrodes, may be coupled directly to pulse generator 108 and sensing and signal processing circuitry 104 without optional switching circuitry 103.

In one embodiment, sensing and signal processing circuitry 104 includes a respiratory activity module 106, a fatigue sensing module 120, a respiration or metabolic status module 122, and a respiration demand module 124. As used herein, the term “module” refers to an application specific integrated circuit (ASIC), an electronic circuit, a processor (shared, dedicated, or group) and memory that execute one or more software or firmware programs, a combinational logic circuit, or other suitable components that provide the described functionality. In addition to electrode signals, sensing unit 104 may be coupled to other physiological sensors 105 which may be coupled to any of the above-mentioned modules.

Sensors 105 may provide signals correlated to respiratory activity, which may be measured when phrenic nerve stimulation is being delivered and/or during periods of no therapy delivery to for comparative analysis of the respiratory response to stimulation at varying settings and for comparing to intrinsic respiratory function. The respiratory response on a respiration cycle may be used to optimize burst stimulation parameters.

Respiratory activity module 106 is provided to measure a response to phrenic nerve stimulation parameters, and may be a measurement taken on a single respiration cycle, e.g. correlated to tidal volume, or multiple respiration cycles. In one embodiment, respiratory activity module 106 is embodied as impedance measuring circuitry provided for measuring thoracic impedance as a signal correlated to respiratory activity. Any available multi-purpose or dedicated electrodes deployed in or around the thorax may be used for delivering a drive current and recording an impedance signal may be used for measuring thoracic impedance. The impedance sensing circuitry includes drive current circuitry and impedance measurement circuitry for monitoring thoracic impedance. Examples of thoracic impedance measurement methods that can be used for monitoring a respiration signal are generally described in U.S. Pat. No. 4,901,725 (Nappholz), U.S. Pat. No. 6,076,015 (Hartley), and U.S. Pat. No. 5,824,029 (Weijand, et al), all of which are hereby incorporated herein by reference in their entirety.

In alternative embodiments, respiratory activity module 106 may include EMG sensing circuitry for monitoring respiratory activity. When present, EMG sensing circuitry receives signals from electrodes 102 placed in operative relation to the diaphragm for sensing EMG signals. EMG signal measurements may be used to verify capture of the diaphragm. EMG signals may be sensed from one or both sides of the diaphragm, and may be used for detecting diaphragm fatigue and/or recovery by fatigue sensing module 120.

In still other embodiments, respiratory activity module 106 may include pressure sensing circuitry receiving a signal from a pressure sensor included in sensors 105 and positioned intra-throracically for determining a measurement of thoracic pressure that is correlated to respiratory activity. In other alternative embodiments, respiratory activity module 106 may include motion sensing circuitry, e.g. an accelerometer, provided for measuring motion associated with respiration, e.g., motion of the lungs, thoracic wall or diaphragm. Other sensors that may be used for monitoring a respiratory response to activation of the diaphragm include an air flow sensor, acoustical sensor, oxygen sensor, strain gauge or other sensors, any of which could be included in sensors 105 and coupled to respiratory activity module 106 for determining a measurement of respiratory activity and/or for use in controlling a respiration therapy.

Respiratory activity measurements obtained by sensing and signal processing circuitry 104 are provided to processing and control 110 for controlling stimulation parameters used by pulse generator 108 in generating trains of stimulation pulses and controlling the timing and delivery of respiration therapy stimulation by pulse generator 108 and electrodes 102. One or more signals sensed by sensors 105 and/or electrodes 102 may be used to determine optimal stimulation control parameters for capturing the diaphragm and producing a desired respiratory response. Respiratory activity measurements may be used to select optimal stimulation electrodes, electrode polarity, and stimulation parameters, including, but not limited to, pulse amplitude, pulse width, pulse number, and frequency of pulses in a pulse train, for achieving a desired effect on respiration caused by phrenic nerve stimulation.

In some embodiments, respiratory activity measurements are used to detect fatigue of the diaphragm. Impedance measuring circuitry or other sensors and corresponding circuitry described above used for monitoring respiratory activity, such as EMG sensing, pressure sensing, and motion sensing, or any combination thereof, may provide a “fatigue signal(s)” for use by processing and control 110 in detecting diaphragm fatigue, or recovery from fatigue. The term “fatigue signal” as used herein refers to any signal that is correlated to fatigue of the diaphragm.

Fatigue sensing module 120 may be configured to receive one or more signals correlated to respiratory activity and for generating one or more parameters from which fatigue can be detected. Processing and control 110 is configured to use a fatigue signal or output from fatigue sensing module 120 for adjusting stimulation rates of a right portion of the diaphragm and a left portion of the diaphragm. In particular, processing and control 100 adjusts a ratio of unilateral to bilateral stimulation rates as will be further described below and controls which side is being stimulated unilaterally during an interval of time.

In response to detecting the onset of fatigue, processing and control 110 adjusts one or more stimulation control parameters controlling stimulation to one or both sides of the diaphragm. In particular, processing and control 110 controls a rate of pulse trains delivered to each of the right diaphragm and left diaphragm during stimulation therapy delivery to reduce diaphragm fatigue. Reducing diaphragm fatigue includes preventing the onset of fatigue from occurring in the first place and/or allowing recovery from fatigue when it has occurred by reducing demand when fatigue is detected. Generally, the onset of fatigue is detected and responded to prior to complete fatigue of the diaphragm muscle, in an effort to preclude fatigue.

Physiological sensors 105 and/or electrodes 102 may be used for detecting fatigue of one or both sides of the diaphragm, separately or as a whole. In some embodiments, a single signal or combination of signals that are correlated to respiratory activity are used to detect fatigue of the diaphragm as a whole, whether the fatigue is occurring in the left, right or both hemidiaphragms.

In other embodiments, sensors 105 and/or electrodes 102 may be configured to generate separate signals correlated to a respiratory response on the right side and a respiratory response on the left side, respectively. The sensors 105 and/or electrodes 102 can be configured to produce a “right-side” signal that is more highly sensitive to activation of the right portion of the diaphragm or respiration of the right lung and a “left-side” signal that is more highly sensitive to activation of the left portion of the diaphragm or respiration of the right lung.

For example, a first pair of EMG electrodes may be positioned for sensing right diaphragm activation and a second pair of EMG electrodes may be positioned for sensing left diaphragm activation. In another example, a pressure sensor or an accelerometer may be positioned for measuring pressure or motion in the right thoracic cavity and a second pressure sensor or accelerometer may be positioned for measuring pressure or motion in the left thoracic cavity.

These separate “right” and “left” signals allow fatigue in the right and left sides of the diaphragm to be determined separately, enabling separate control of the stimulation delivered to each of the right and left sides to reduce fatigue. In particular, the rate of diaphragm contractions induced by pulse trains delivered to a right or left phrenic nerve or diaphragm is controlled to produce a rate of contractions on each side that reduces diaphragm fatigue. The signals and measurements used for detecting fatigue on the right side and on the left side independently may be the same type of signals and measurements, e.g. a right EMG signal and a left EMG signal or a right pressure signal and a left pressure signal with corresponding measurements derived there from. Alternatively, the right and left fatigue signals may be different types of signals. For example, a right EMG signal may be used to detect right diaphragm fatigue and a left pressure signal may be used to detect left diaphragm fatigue.

While it may be desirable to detect fatigue using the same type of signals acquired from each side of the patient's body, obtaining the same type of signals from both sides of the patient's body for obtaining independent right and left fatigue signals may not always be practical depending on the sensors available and the particular IMD system configuration. For example, if an IMD is implanted in an abdominal pocket on the right side of the patient, electrodes on the IMD housing and/or carried by extravascular leads extending from the IMD may be used to measure a right diaphragm EMG signal while electrodes carried by a transvenous lead may be positioned for measuring a left thoracic impedance signal. As such, detecting fatigue in the right and left sides of the diaphragm separately as described herein does not necessarily require sensing the same type of signal on the right and left sides of the patient's body.

In addition to monitoring induced or intrinsic respiratory activity, sensing and signal processing circuitry 104 may receive signals from sensors 105 and/or electrodes 102 for determining one or more parameters correlated to a respiration demand. Sensing and signal processing circuitry 104 is shown to include a respiration demand module 124 for generating a parameter from one or more sensed signals correlated to respiratory demand and used to establish an intended induced respiration rate.

Processing and control may receive sensor signals directly for establishing a respiration rate or receive processed parameters from sensing and signal processing circuitry 104. The respiration rate may be established based on a heart rate, patient activity level, patient posture, an oxygen saturation measurement, other signals correlated to the metabolic demand of the patient, or any combination thereof. In some embodiments a respiration rate is established as a user-entered programmed parameter and may be set based on time of day.

Sensing and signal processing circuitry 104 may further include a module 122 for determining a status of the respiration function or the metabolic status of the patient. This status reflects how well the stimulation therapy is meeting a targeted respiratory function or targeted metabolic parameter value. For example, a targeted range for minute ventilation may be established by user input or based at least in part on the respiration demand. Processing and control 110 may adjust stimulation parameters to maintain the minute ventilation within a targeted range. In particular a frequency of bilateral activation of the diaphragm may be adjusted during a time period of alternation between bilateral and unilateral activation as will be described further herein. A respiration function status, such as minute ventilation, determined by module 122 reflects respiratory activity over multiple respiration cycles in contrast to a single respiration cycle measurement such as tidal volume. Minute ventilation may be determined using thoracic impedance signals in one embodiment.

A metabolic status may be measured as a blood chemistry parameter, such as venous pH and/or venous blood oxygen saturation or blood conductivity. A targeted range may be established for maintaining a normal metabolic status for a given patient in which respiration therapy is used to support the patient in meeting his/her metabolic demand. Processing and control 110 will adjust the respiration therapy to maintain the metabolic status parameter within an established, acceptable range.

Processing and control 110 receives signals from sensing and signal processing 104 and may receive signals directly from sensors 105. In response to received signals, processing and control 110 controls delivery of phrenic nerve stimulation by pulse generator 108. Received signals may additionally include user command signals received by communication circuitry 114 from an external programming device or data acquired from patient images received from an external imaging device such as a fluoroscopy unit.

Processing and control 110 may be embodied as a programmable microprocessor and associated memory 112. Processing and control 110 and sensing and signal processing circuitry 104 may be implemented as any combination of an application specific integrated circuit (ASIC), an electronic circuit, a processor (shared, dedicated, or group) and memory that execute one or more software or firmware programs, a combinational logic circuit, or other suitable components that provide the described functionality.

Memory 112 stores parameters and algorithms used by processing and control 110. Memory 112 may additionally store data associated with sensed signals and delivered respiration therapy. Data may be transmitted to an external device by communication circuit 114, which typically includes wireless transmitting and receiving circuitry and an associated antenna for bidirectional communication with an external device. Processing and control 110 may generate reports or alerts that are transmitted by communication circuitry 114.

Alert circuitry 116 may be included for generating a patient alert signal to notify the patient of a condition warranting medical attention. The patient may be alerted via an audible sound, perceptible vibration, IMD pocket stimulation, or the like and be advised to seek medical attention upon perceiving an alert signal.

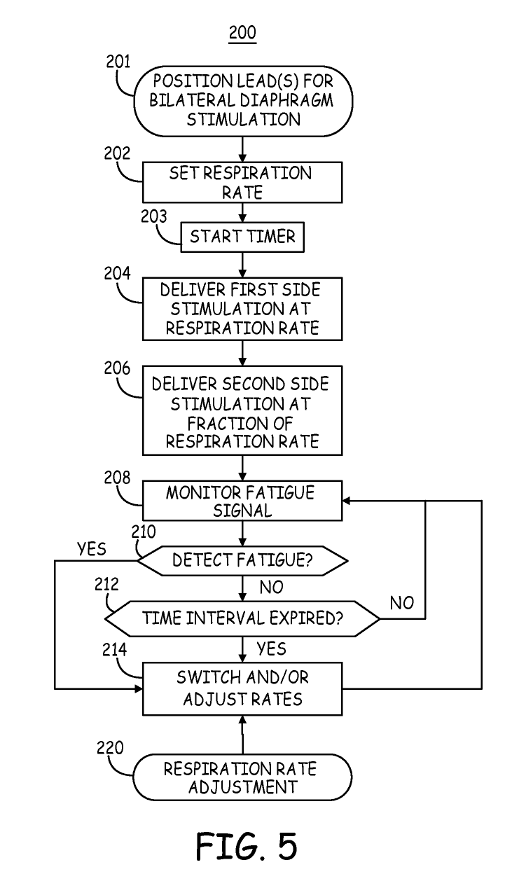

FIG. 5 is a flow chart 200 of a method for controlling bilateral phrenic nerve stimulation for delivering a respiration therapy according to one embodiment. Flow chart 200 is intended to illustrate the functional operation of the device, and should not be construed as reflective of a specific form of software or hardware necessary to practice the methods described. It is believed that the particular form of software, hardware and firmware used in an IMD system will be determined primarily by the particular system architecture employed in the device and by the particular sensing and therapy delivery methodologies employed by the device. Providing software, hardware and/or firmware to accomplish the described functionality in the context of any modern IMD, given the disclosure herein, is within the abilities of one of skill in the art.

Methods described in conjunction with flow chart 200 may be implemented, at least in part, in a computer-readable medium that includes instructions for causing a programmable processor to carry out the methods described. A “computer-readable medium” includes but is not limited to any volatile or non-volatile media, such as a RAM, ROM, CD-ROM, NVRAM, EEPROM, flash memory, and the like. The instructions may be implemented as one or more software modules, which may be executed by themselves or in combination with other software.

At block 201, one or more leads may be positioned for delivering bilateral diaphragm stimulation. Illustrative embodiments presented herein refer primarily to bilateral phrenic nerve stimulation. Any of the transvenous phrenic nerve stimulation leads and electrode configurations shown in FIGS. 1 through 3 may be used. Alternative embodiments, however, may include direct or indirect phrenic nerve and/or diaphragm stimulation in any combination to achieve bilateral diaphragm stimulation.

At block 202, a respiration rate is set. A respiration rate may be a baseline respiration rate to be induced by stimulating the diaphragm (directly or via the phrenic nerves) according to a respiration therapy protocol. The rate may be set above a patient's intrinsic rate or set at a rate determined using a sensor signal correlated to metabolic need. The respiration rate may be a rate delivered twenty-four hours per day for full respiratory support, periodically to augment intrinsic respiratory activity, during the night only for treating apnea or other breathing disorders, or upon detecting a need for respiratory support in response to a sensed or received signal. Individual patient need and the primary indication for respiratory therapy will dictate the type of respiration therapy protocol used and how often during the day, night, week, etc. respiration therapy is delivered.

At block 203, a timer is started. The timer may be set to a maximum time interval for delivering a particular stimulation rate to one side of the diaphragm before altering a stimulation rate of that side. Stimulation pulse trains are delivered to one side of the diaphragm at the intended induced respiration rate at block 204. To reduce fatigue, stimulation pulse trains are delivered to the other side of the diaphragm at a rate less than the respiration rate. The individual stimulation pulse trains delivered to the right and left sides may be equivalent, i.e, delivered with the same pulse energy, pulse number, frequency of pulses within the pulse train, pulse train duration, etc., but are delivered at different rates to cause alternation between unilateral and bilateral activation of the right and left sides of the diaphragm. Alternatively, the right and left pulse trains may be defined distinctly from each other according to parameters defining a pulse train found to be optimal for capturing the respective side of the diaphragm and eliciting a desired respiratory response. In either case, the left and right sides are activated at different rates to cause an alternation between unilateral and bilateral stimulation during a given time interval.

At block 208, a fatigue signal is monitored. A fatigue signal may be any signal correlated to a respiratory response to the stimulation pulse trains. Any of the sensors and signals described above may be used to detect fatigue at block 210, by detecting, for example, a decrease or decreasing trend in the signal. In some cases, detecting fatigue may include detecting a loss of capture of the diaphragm from the signal or an increase in stimulation threshold.

If fatigue is not detected, and the time interval has not expired as determined at block 212, the stimulation continues at the current rates for the right and left sides. If fatigue is detected, or the time interval expires, the rates of delivering the stimulation pulses to the right and left sides are adjusted at block 214. In one embodiment, the rates are switched such that if the left side was initially stimulated at the respiration rate and the right side was initially stimulated at a proportion of the respiration rate, e.g. one-half or one-third the respiration rate, the rates may be switched such that the right side is stimulated at the intended respiration rate and the left side is stimulated at the proportion of the respiration rate, or a new proportion of the respiration rate.

The rates delivered to the right and left sides may be adjusted depending at least in part on whether fatigue was detected. For example, if fatigue is being detected, the first side being stimulated at the respiration rate may be slowed to a new proportion of the respiration rate that is less than the proportion initially delivered to the second side. The second side is increased to the intended respiration rate to maintain the intended respiration rate while the first side recovers.

The time interval controlling the time at which a rate adjustment is made may be predetermined and may be dependent on the current respiration rate. For example, the time interval may be one minute, two minutes, five minutes, thirty minutes, one hour or another interval. During slower respiration rates, for example when the patient is resting, the time interval may be set a relatively longer interval, and during faster respiration rates, for example when the patient is exercising, the time interval may be set to a relatively shorter interval to reduce fatigue in the side being stimulated at the intended respiration rate.

Furthermore, the respiration rate may be adjusted at any time during a given time interval, as indicated by block 220. Physiological sensors may be used to detect a need for increased (or decreased) respiration as described above. A closed-loop feedback algorithm may be used to sense the need for respiration and adjust the respiration rate automatically. For example, an activity sensor, posture sensor, oxygen sensor, pH sensor, temperature sensor, hemodynamic sensor, or any combination thereof, may be used in determining a sensor-indicated respiration rate.

If the respiration rate is to be adjusted, the stimulation rate of one side is increased or decreased accordingly to maintain stimulation of at least one portion of the diaphragm at the respiration rate. The stimulation rate of the other side of the diaphragm may remain at a previously set proportion of the respiration rate as the respiration rate is adjusted. Alternatively the proportion of the respiration rate at which the second side is stimulated may also be adjusted if the respiration rate is adjusted.

In an illustrative example, the right side is stimulated at the respiration rate, and the left side is stimulated at half the respiration rate. Fatigue is detected in response to a fatigue signal. The left side is stimulated at the respiration rate and the right side is stimulated at half the respiration rate in response to detecting fatigue. A need for an increase in respiration rate is determined in response to an activity signal or other metabolic-related signal. The right side is increased to a higher respiration rate while the rate proportion delivered to the left side is decreased due to the recent detection of fatigue when the left side was being stimulated at the respiration rate. The decreased proportion may be in relation to the proportionate increase in the respiration rate to maintain a lower rate applied to the left side.

In some embodiments, the fatigue signal may additionally be monitored for recovery to allow a rate proportion to be increased as recovery is detected. As described above, separate fatigue signals may be monitored for detecting right and left sided fatigue of the diaphragm and allowing rate adjustment to the two sides accordingly.

While the flow chart 200 includes blocks indicating actions or decisions taken in a particular order, it is recognized that the blocks shown in flow chart 200 may be performed in a different order than that shown. Furthermore, some actions or decisions may be omitted and control of a respiration therapy may still be performed in a manner that successfully reduces diaphragm fatigue. For example, fatigue signal monitoring is not required in some embodiments. Rate adjustments may be performed on a scheduled basis to reduce fatigue without monitoring a fatigue signal. In other embodiments, a fixed respiration rate may be set without including respiration rate adjustments.

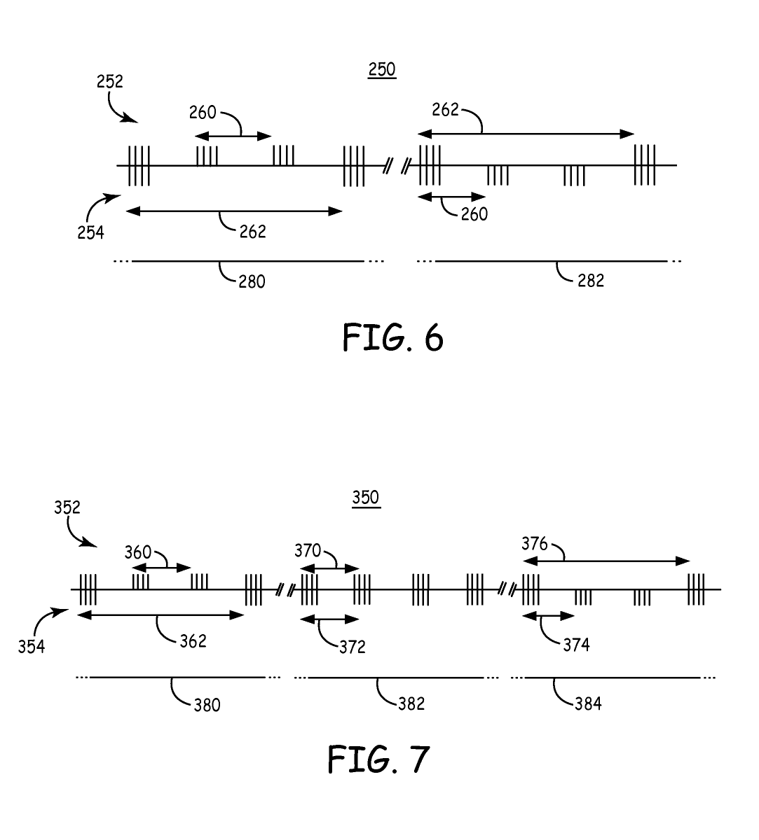

FIG. 6 is a timeline 250 showing pulse trains 252 delivered to activate a first side of the diaphragm, e.g. to the left phrenic nerve, and pulse trains 254 delivered to activate the other side of the diaphragm, e.g. to the right phrenic nerve. Initially pulse trains 252 are delivered to the left side at a rate, as defined by pulse train interval 260, corresponding to a respiration rate intended to be induced by the nerve stimulation therapy.

The stimulation pulse trains 254 delivered to the right side are shown to be delivered at pulse train interval 262 that is approximately three times longer the pulse train interval 260, resulting in activation of the right diaphragm at one-third the rate of the left diaphragm. As can be seen in FIG. 6, the left and right pulse trains 252 and 254 are delivered in a substantially synchronous manner during every respiration cycle in which the right side is stimulated. In other words, the side stimulated at the slower rate is not stimulated alone, in a unilateral manner, but is stimulated in synchrony with the left side in a bilateral manner, whenever it is stimulated. In this way, the stimulation therapy alternates between unilateral and bilateral stimulation over repeatedly over a given time interval 280, for example over one minute.

Such unilateral-bilateral alternation may be expressed as a ratio of the first side rate to the second side rate, such as a 3:1 rate ratio (or vice versa as 1:3) as shown in FIG. 6. Alternatively, the unilateral-bilateral alternation may be expressed as a ratio of bilateral stimulation cycles to unilateral stimulation cycles. For example, a 1:2 ratio would be one bilateral stimulation cycle for every 2 unilateral cycles or R+L, R, R. This stimulation pattern of a 1:2 bilateral to unilateral ratio would be expressed as a ratio of right side rate to left side rate of 3:1 (3 right side activations for every one left side activations).

Alternating stimulation between the right and left sides in a unilateral manner, e.g. R-L-R-L or R-R-R-L-L-L, would result in neither side being delivered at an intended respiration rate over a complete period of the alternating pattern. In contrast, throughout a complete period of a unilateral-bilateral alternation pattern one side is stimulated at the intended respiration rate and the other side at a proportion of the respiration rate, e.g. (R+L)-L-L-(R+L)-L-L and so on as shown in FIG. 6. The left side is stimulated at the respiration rate throughout a complete period of the stimulation pattern (R+L)-L-L and this pattern is repeated over a given time interval or until fatigue is detected.

The ratio of the rates applied to one side and to the other side to achieve alternating bilateral and unilateral simulation may be any of 2:1, 3:1, 4:1, 3:2, 4:3, 5:2, 5:3, or any other ratio. When the left and right phrenic nerves are both stimulated during a respiration cycle, the pulse trains may be aligned in time or one pulse train may be somewhat delayed relative to the other pulse train. As such, synchronous right and left stimulation during a given respiration cycle does not necessarily require simultaneous pulse trains but rather pulse trains that are both delivered during the given respiration cycle.

After an predetermined interval of time 280, or upon detecting diaphragm fatigue, which may be primarily left-side fatigue when the left side is stimulated at the respiration rate, the stimulation rates are switched such that the left side is stimulated at a proportion of the induced respiration rate, e.g., corresponding to the pulse train interval 262, and the right side is stimulated at the respiration rate, e.g., corresponding to the pulse train interval 260. A new time interval 282 is started. The pulse train interval 260 setting the induced respiration rate may be adjusted at any time in response to a sensed signal used to determine a needed respiration rate. Likewise, the pulse train interval 262 may be increased or decreased in response to an adjustment to interval 260.

If a higher respiration rate is needed during a time interval 282, the interval 260 is shortened to increase the rate of activating the right diaphragm. The interval 262 may be kept substantially the same and adjusted only as needed to maintain synchronous stimulation on bilateral cycles during the new respiration rate. The rate of bilateral stimulation cycles per minute may remain approximately the same depending on how much the respiration rate is adjusted.

Alternatively, interval 262 may be adjusted to be shorter or longer in response to a change in interval 260, such that the ratio between the stimulation rates of the right and left sides is either increased (resulting in more frequent bilateral stimulation) or decreased (resulting in less frequent bilateral stimulation). In other words, the rate proportion applied to the left side during interval 282 may be adjusted in response to a change in the induced respiration rate, i.e. interval 260 applied to the right side. The decision whether to adjust the pulse train interval 262 may depend at least in part on whether fatigue was recently detected in association with the left side prior to time interval 282.

Furthermore the predetermined time intervals 280 and 282 may be adjusted in response to changing the pulse train interval 260 that sets the induced respiration rate. If the respiration rate is increased by shortening interval 260, a timed interval 280 or 282 may be adjusted to a shorter time interval to avoid stimulation of one side of the diaphragm at the higher respiration rate for a prolonged period. If the respiration rate is decreased by lengthening interval 260, the time interval 280 or 282 may be increased.

If fatigue is detected during interval 280 or 282, the pulse train intervals 260 and 262 may be adjusted prior to expiration of the time interval 280 or 282 to reduce fatigue. For example, if fatigue is detected during interval 280, interval 280 is ended prematurely, and a new time interval 282 is started upon adjusting the rates due to fatigue. The left side is stimulated at the pulse train interval 262, or another longer interval, and the right side is stimulated at the interval 260 corresponding to an intended induced respiration rate.

Time interval 282 may also be adjusted based on whether or not fatigue was detected during the preceding time interval 280 causing it to be terminated prematurely. For example, time interval 282 may be lengthened if no fatigue is detected during interval 280. At the current respiration rate, one side may be able to sustain the rate for a somewhat longer period of time without fatigue. Time interval 282 may be shortened if fatigue is detected during interval 280. For a given respiration rate, interval 280 may be too long to maintain one side at that respiration rate resulting in fatigue. In this way, the time intervals 280, 282 set for maintaining a respiration rate on one side of the diaphragm can be “learned” over time for different intended respiration rates, based on whether fatigue is detected or not.

FIG. 7 is a timeline 350 illustrating an alternative method for controlling activation of the diaphragm to reduce fatigue. Stimulation pulse trains 352 are delivered to a first side, for example the left side, during a time interval 380 at an pulse train interval 360 corresponding to an intended respiration rate. Stimulation pulses 354 are delivered to a second side, for example the right side, at a fraction of the intended respiration rate, for example at one-third the rate, corresponding to an pulse train interval 362.

If fatigue is not detected, or if a need for a stronger respiratory response is detected, the stimulation rate of the second side may be increased for an interval of time 382, to a larger fraction of the intended respiration rate or to a rate that is equal to the intended respiration rate by stimulating the second side synchronously with the first side during each respiration cycle. The two inter-pulse intervals 370 and 372 are substantially equal and correspond to an induced respiration rate. Alternatively, the rate proportion applied to the second side may be increased, e.g. from a ratio of 1:3 during time interval 380 to a ratio of 2:3 or other higher ratio resulting in an increased frequency of bilateral stimulation during time interval 382.

When bilateral stimulation is delivered during the interval of time 382, other stimulation parameters may be alternated to reduce diaphragm fatigue. For example, stimulation pulse amplitude, pulse width, pulse number or pulse frequency may be alternated from cycle-to-cycle, either between the left and right sides or from cycle-to-cycle on a given side. For example, the stimulation pulse amplitude may be delivered at a high level for two beats on the right and a low level for the same two beats on the left then alternated to deliver a low level amplitude for the next two beats on the right while the left side is stimulated at a higher level amplitude for those same two beats. Such alternation may occur at any desired ratio or period.

In another example, the stimulation parameters may remain the same on one side throughout a time interval of bilateral stimulation while the stimulation parameters alternate from cycle-to-cycle on the other side. If the left side is being stimulated at a given set of stimulation parameters on every respiration cycle during bilateral stimulation, the right side may be stimulated synchronously with the left side at the respiration rate but with alternating pulse amplitude from cycle-to-cycle, e.g. 2V, 5V, 5V, 2V, 5V, 5V . . . etc., or another alternating stimulation parameter (pulse number, pulse frequency, etc.)

Upon expiration of a predetermined time interval 382 or upon detection of diaphragm fatigue or a reduced need for respiratory support, the stimulation is adjusted to set the right side stimulation pulse trains to occur at a pulse train interval 374 corresponding to an intended respiration rate for a time interval 384. The left side stimulation pulse trains are adjusted to a pulse rate corresponding to interval 376 that corresponds to a proportion of the intended respiration rate, in this example one-third.

In various embodiments, activation of the diaphragm is delivered in intervals of time in which one side is stimulated at an intended induced respiration rate and on the other side is stimulated at a proportion of the intended induced respiration rate that is slower than the respiration rate. These intervals may be separated by intervening intervals of time during which stimulation is delivered at equal rates to the right and left phrenic nerves and/or intervals of time during which no stimulation is delivered to the right and left phrenic nerves.

Within a given time interval, the stimulation alternates between unilateral and bilateral stimulation. In other words, stimulation is not alternating between the left and right sides but is alternating between unilateral and bilateral stimulation in that a first side is activated on all respiration cycles and the second side is activated on a proportion of, i.e. fewer number of, all respiration cycles.

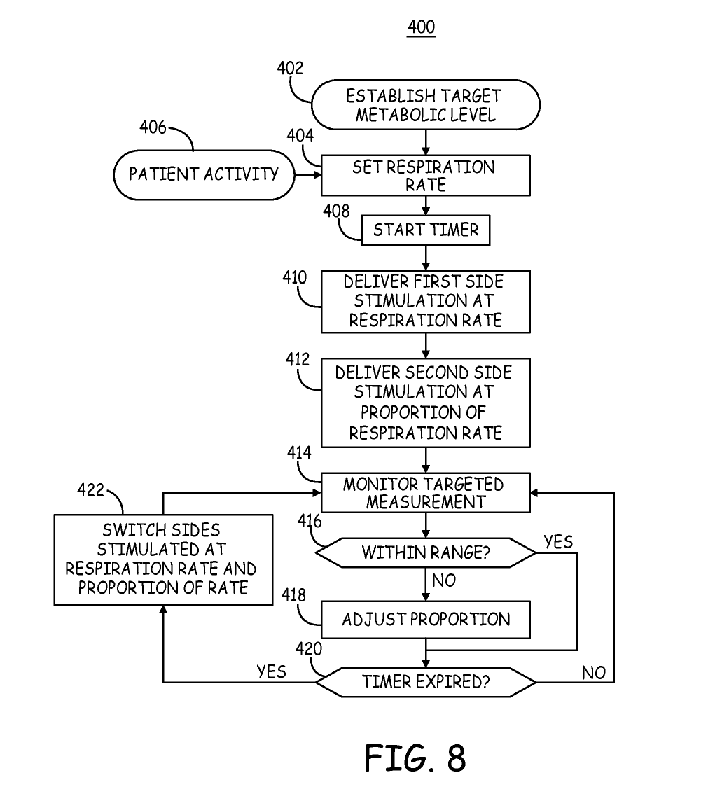

FIG. 8 is a flow chart 400 of an alternative method for controlling bilateral activation of the diaphragm. At block 402, a targeted metabolic or respiration parameter value range is established. This may be a targeted minute ventilation, a targeted venous pH, a targeted blood conductivity, targeted venous blood oxygen saturation, or any combination thereof. The metabolic parameter or respiration parameter is a measurement that represents the cumulative effects of a respiration rate and tidal volume over multiple respiration cycles as opposed to respiratory activity measured on a single respiration cycle. The targeted range may be established based on user input and tailored for a particular patient. The targeted range may vary with other factors, such as heart rate and patient activity.

At block 404, a respiration rate is established and set. The respiration rate may be set based on patient activity level input at block 406. Alternatively, a respiration rate may be established as described previously based on other sensor input, such as a posture sensor, heart rate or any combination of activity, posture and heart rate or other indicators of the need for respiration or metabolic demand. At block 408, a timer is started to control a maximum time interval for which a first side is stimulated at the established respiration rate at block 410. The second side is stimulated at a proportion of the respiration rate at block 412.

At block 414, the targeted measurement which is correlated to a metabolic or respiratory status of the patient, is monitored. In one embodiment, minute ventilation, venous blood pH, venous blood oxygen saturation, blood conductivity, or any combination thereof is monitored. If the measurement falls within an acceptable range of the target, as determined at block 416, and the timer has not yet expired, the stimulation rates applied to the first and second sides are maintained and the process continues to monitor the targeted measurement.

If the measurement is outside the acceptable target range, the proportion at which the second side is stimulated at is adjusted at block 418. The proportion may be increased to cause more frequent respiration cycles in which both the right and left side of the diaphragm are activated in a bilateral manner. The proportion may be decreased to cause less frequent bilateral activation of the diaphragm. By adjusting the proportion to cause a different frequency of bilateral activation during the time interval, the respiratory response can be carefully titrated to maintain the minute ventilation of the patient at a level that achieves a targeted range of a respiratory or metabolic parameter. In some embodiments, minute ventilation may be measured and maintained within a target value and/or a metabolic related measurement, such as venous blood pH, blood conductivity, or venous oxygen saturation, is measured and maintained within a target value.

If the timer expires, as determined at block 420, the side being stimulated at the respiration rate is switched at block 422. The first side is stimulated at a proportion of the respiration rate to reduce fatigue of the first side. The second side is stimulated at the respiration rate. While not explicitly shown in FIG. 8, other aspects described above may be included in the method shown in FIG. 8 such as monitoring a signal to detect fatigue and cause early termination of the time interval. A subsequent time interval may be adjusted in response to detecting fatigue

Additionally or alternatively, the respiration rate may be adjusted any time during a given time interval to maintain a desired respiration rate based on a respiration demand signal. The rate proportion applied to the phrenic nerve being stimulated slower than the respiration rate may be adjusted in response to the respiration rate adjustment. The timer controlling the time interval may be adjusted in response to adjusting a respiration rate.

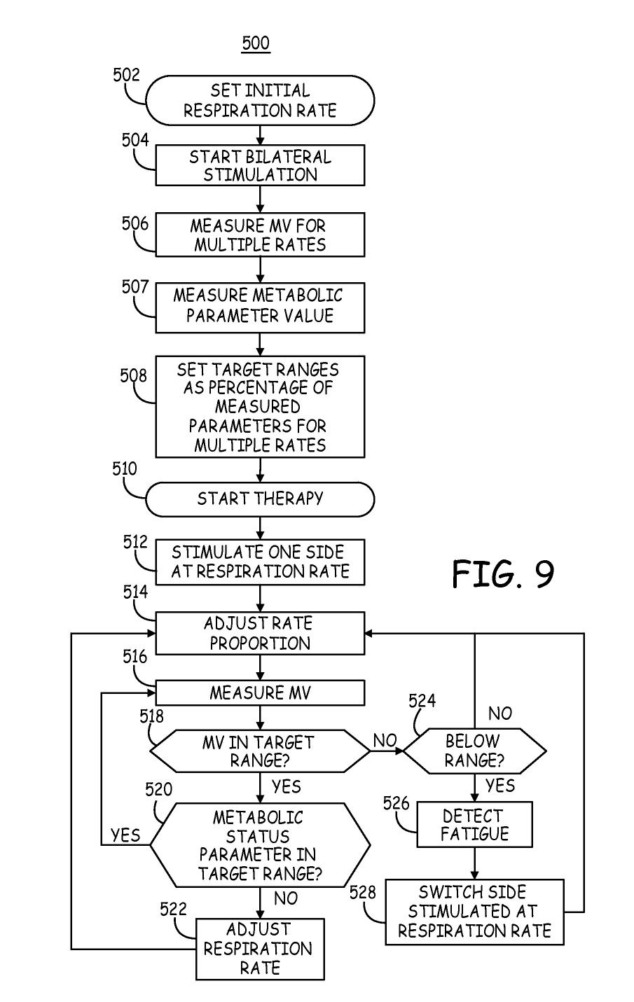

FIG. 9 is a flow chart 500 of a method for establishing a target range for a respiratory status parameter and controlling alternation of bilateral and unilateral activation of the diaphragm according to one embodiment. At the time of implantation of an IMD system or at follow up visits, a target range for a respiratory status parameter may be established during a testing procedure, prior to enabling the respiration therapy. To begin the testing procedure, an initial respiration rate is set at block 502. Bilateral stimulation is enabled at a 1:1 ratio at block 504, i.e., bilateral activation of the diaphragm on every respiration cycle.

During bilateral stimulation at the 1:1 ratio, a respiratory status parameter is measured at block 506. In the illustrative embodiment, the respiratory status parameter is minute ventilation (MV) though other parameters could be measured that take into account respiratory function over multiple respiration cycles or over a predefined time interval such as thirty seconds or more. This measurement provides a basis for establishing a target range of the parameter during alternating unilateral-bilateral stimulation. The stimulation pulse trains are delivered at a supra-threshold pulse amplitude and may be delivered at some maximum pulse number and pulse frequency within the pulse train to cause a maximal contraction of both the right and left portions of the diaphragm.

The measured minute ventilation represents a maximum respiratory function at the delivered respiration rate. This maximum function may represent a maximum breathing effort that will lead to diaphragm fatigue when sustained. As such, a targeted range of the respiratory status parameter is set as a fraction or percentage of this maximum parameter value to reduce fatigue.

The respiratory status parameter is measured for multiple respiration rates at block 506, using supra-threshold bilateral stimulation at all rates to obtain the maximum respiratory function at each rate. After measuring MV for multiple respiration rates, a minimum value or target range for MV is set for each of the multiple respiration rates at block 508 as a percentage of the maximum MV measured for a given rate. Since tidal volume may vary between respiration rates, different target ranges may be set for different respiration rates. In one embodiment, the target range is set at eighty percent of the maximum value, plus or minus five percent. Other percentages may be selected based on individual patient need. In alternative embodiments, a maximum MV may be measured at a predetermined minimum respiration rate and a maximum respiration rate and target ranges for intermediate respiration rates may be interpolated using a linear or non-linear relationship.

If a supra-threshold pulse train is delivered to activate the diaphragm, adjustments to pulse train parameters, such as pulse amplitude, pulse number, and pulse train frequency will have small effects on the tidal volume on a give respiration cycle. As such, adjustment of the pulse train parameters may have limited effects on an overall respiratory status parameter such as minute ventilation. For example, if a pulse train having a pulse number and pulse frequency that produces a fused titanic contraction of the diaphragm, adjustment of pulse amplitude greater than the stimulation threshold may have little change on the tidal volume on a given breath. Relatively small changes in tidal volume will have limited effects on the minute ventilation or other parameter that measures the respiratory status over multiple respiration cycles. By controlling the ratio of unilateral to bilateral stimulation, greater control over the titration of the respiratory status parameter of the patient can be achieved. This control of a ratio of unilateral and bilateral stimulation will allow some minimum acceptable level of the respiratory status (e.g. minute ventilation) to be maintained while allowing maximum frequency of unilateral respiration cycles to reduce fatigue and still meet the metabolic demand of the patient.

A metabolic parameter value may be measured at block 507. The metabolic parameter value will indicate whether the respiratory function is meeting the metabolic demand of the patient. For example, venous pH may be measured at block 507 and may optionally be compared to the minute ventilation to determine a relationship between venous pH and the maximum MV measured at each respiration rate. A target range for the venous pH for a given respiration rate may be established based on these measurements at block 508, which may result in the same or different target ranges at different respiration rates. Alternatively a target range for a metabolic status parameter may be based on clinical data and may be the same for all respiration rates. In some embodiments, the minute ventilation and venous blood pH may be measured at different respiration rates during corresponding different levels of patient activity, e.g. rest, sub-maximal exercise, and maximal exercise.

Once the target ranges for the respiratory status parameter and metabolic status parameter are established for multiple respiration rates, the therapy is started at block 510. During therapy delivery, one side of the diaphragm is activated at the respiration rate at block 512. Initially the respiration rate may be set to a default value and will be subsequently adjusted based on an indicator of metabolic demand. At block 514, the other side of the diaphragm is stimulated at a proportion of the respiration rate, which may be an initial default rate proportion, e.g. every second, third or fourth respiration cycle.

At block 516, the minute ventilation is measured and compared at block 518 to the target range established for the current respiration rate. If the MV is within the target range, the metabolic status parameter is compared to its target range at block 520. If the metabolic status parameter is outside the target range, the respiration rate is adjusted at block 522. As such, in some embodiments, the metabolic status parameter may be used as a metabolic demand signal for adjusting the intended induced respiration rate. Alternatively, the frequency of bilateral stimulation may be increased by increasing the rate proportion applied to the other side of the diaphragm first to determine if the metabolic status parameter can be brought back into range before adjusting the respiration rate.

If the respiration rate is adjusted at block 522, the process returns to block 514 to adjust the rate proportion applied to the other side, which may be an increase, decrease, or not change to the rate proportion, as described previously herein.

If the MV is outside the target range at block 518, but is above the targeted range (negative result at decision block 524), the rate proportion may be adjusted to reduce the frequency of bilateral activation of the diaphragm at block 514. This allows greater rest to the other side of the diaphragm to reduce the likelihood of fatigue while maintaining the target range of the respiratory status parameter.

If the MV is below the target range at block 524, diaphragm fatigue is detected at block 526. As such, a respiratory status parameter may be used as a fatigue signal for controlling the time intervals at which a first side is stimulated at the respiration rate. At block 528, the stimulation rate applied to the side being stimulated at the rate proportion is adjusted to the intended respiration rate, and the side being stimulated at the respiration rate (presumably presenting fatigue) is adjusted to a rate proportion. While not shown in FIG. 9, it is to be understood that a timed interval may also be used in conjunction with a fatigue signal to control the time intervals and rate proportions.

The rate proportion may be adjusted to a lower proportion to reduce the frequency of bilaterally activated respiration cycles at block 514 to allow greater rest to the side presenting fatigue. In this way, both a respiratory status parameter and a metabolic status parameter are used to cooperatively control respiration rate applied to one side of the diaphragm, the rate proportion applied to the other side of the diaphragm, and the switching of the rates between the two side to reduce fatigue, maintain a desired respiratory function and a desired metabolic status. The process shown by flow chart 500 illustrates that the control processor of an IMD may use common signals (e.g. a thoracic impedance signal and venous blood pH signal) to establish a respiration rate, detect fatigue, establish and monitor a respiratory status parameter, and establish and monitor a metabolic status parameter.

Thus, a method and apparatus for providing a respiration therapy have been presented in the foregoing description with reference to specific embodiments. It is appreciated that various modifications to the referenced embodiments may be made without departing from the scope of the disclosure as set forth in the following claims.

1. A method for delivering a respiration therapy, comprising:
establishing an intended induced respiration rate;
setting a timer for a first time interval;
delivering a first plurality of stimulation pulse trains to activate a first portion of a patient's diaphragm at the intended respiration rate during the first time interval; and
delivering a second plurality of stimulation pulse trains to activate a second portion of a patient's diaphragm at a second rate during the first time interval, the second rate being different than the intended respiration rate.

2. The method of claim 1, wherein the second rate is a proportion of the induced respiration rate and delivery of the second plurality of stimulation pulse trains at the second rate causes repeated alternation between unilateral and bilateral diaphragm activation during the first time interval.

3. The method of claim 1 further comprising:
monitoring a signal correlated to diaphragm fatigue;
detecting fatigue in response to the fatigue signal;
responsive to detecting fatigue terminating the time interval prematurely and starting a next time interval;
stimulating the first portion at a proportion of the intended induced respiration rate during the next time interval; and
stimulating the second portion at the respiration rate during the next time interval.

4. The method of claim 3 further comprising adjusting the next time interval relative to the first time interval in response to detecting fatigue.

5. The method of claim 3 wherein monitoring the signal correlated to diaphragm fatigue comprises monitoring a first signal corresponding to fatigue of the first portion of the diaphragm and monitoring a second signal corresponding to fatigue of the second portion of the diaphragm.

6. The method of claim 1, comprising:
monitoring a signal correlated to respiratory demand; and
adjusting the respiration rate during the first time interval in response to the respiratory demand.

7. The method of claim 6, comprising adjusting the first time interval in response to adjusting the respiration rate.

8. The method of claim 6 further comprising adjusting the second rate in response to adjusting the respiration rate.

9. The method of claim 1 further comprising:
establishing a targeted range of at least one of a metabolic parameter and a respiration parameter;
monitoring the at least one parameter during the first time interval; and
adjusting the second rate in response to the parameter not falling within the targeted range.

10. The method of claim 9 wherein establishing the targeted range comprises measuring the at least one parameter during bilateral activation of the diaphragm.

11. A respiration therapy delivery system, comprising:
a plurality of electrodes;
a pulse generator coupled to the plurality of electrodes;
a timer for controlling a first time interval; and
a control processor configured to control the pulse generator to deliver a first plurality of stimulation pulse trains to activate a first portion of a patient's diaphragm at an intended induced respiration rate during the first time interval, and
deliver a second plurality of stimulation pulse trains to activate a second portion of a patient's diaphragm at a second rate during the first time interval, the second rate being different than the intended respiration rate.

12. The system of claim 11, wherein the second rate is a proportion of the induced respiration rate and delivery of the second plurality of stimulation pulse trains at the second rate causes repeated alternation between unilateral and bilateral diaphragm activation during the first time interval.

13. The system of claim 11 further comprising a sensor for providing a signal correlated to diaphragm fatigue,
the control processor further configured to
detect fatigue in response to the fatigue signal,
responsive to detecting fatigue cause the timer to terminate the time interval prematurely and start a next time interval, and
control the pulse generator to stimulate the first portion at a proportion of the intended induced respiration rate during the next time interval and stimulate the second portion at the respiration rate during the next time interval.

14. The system of claim 13 wherein the control processor is configured to adjust the next time interval relative to the first time interval in response to detecting fatigue.

15. The system of claim 13 wherein the sensor providing a signal correlated to diaphragm fatigue comprises a first sensor providing a first signal corresponding to fatigue of the first portion of the diaphragm and a second sensor providing a second signal corresponding to fatigue of the second portion of the diaphragm.

16. The system of claim 11, further comprising a sensor providing a signal correlated to a respiratory demand of the patient,
the controller processor configured to adjust the respiration rate during the first time interval in response to the signal correlated to the respiratory demand.

17. The system of claim 16, wherein the control processor is further configured to adjust the first time interval in response to adjusting the respiration rate.

18. The system of claim 16 wherein the control processor is further configured to adjust the second rate in response to adjusting the respiration rate.

19. The system of claim 11 further comprising at least one sensor providing a signal correlated one of a metabolic status and a respiration status of the patient;
the control processor configured to establish a targeted range of a parameter measured from the signal, measure the parameter from the signal, compare the measured parameter to the targeted range, and adjust the second rate in response to the measured parameter not falling within the targeted range.

20. The system of claim 19 wherein establishing the targeted range comprises measuring the parameter during bilateral activation of the diaphragm.

21. A computer-readable medium storing a set of instructions which cause a medical device system to perform a method, the method comprising:
establishing an intended induced respiration rate;
setting a timer for a first time interval;
delivering a first plurality of stimulation pulse trains to activate a first portion of a patient's diaphragm at the intended respiration rate during the first time interval; and
delivering a second plurality of stimulation pulse trains to activate a second portion of a patient's diaphragm at a second rate during the first time interval, the second rate being different than the intended respiration rate.
